(19) United States
(12) Patent Application Publication
(10) Pub. No.: US 2016/0263217 A1
(43) Pub. Date: Sep. 15, 2016

(54) PROTEIN-SPECIFIC FORMULATIONS

(71) Applicant: ENSISHEIM PARTNERS LLC, Seattle, WA (US)

(72) Inventors: Tara MARTIN-CHEN, Seattle, WA (US); Shu-Chih CHEN, Seattle, WA (US); Steven C. QUAY, Seattle, WA (US)

(21) Appl. No.: 15/029,431

(22) PCT Filed: Oct. 16, 2014
(86) PCT No.: PCT/US2014/060954

§ 371 Date: Apr. 14, 2016

Related U.S. Application Data
(60) Provisional application No. 61/891,868, filed on Oct. 16, 2013.

(51) Int. Cl. A61K 39/395; A61K 38/39; A61K 38/42; A61K 9/00; A61K 31/198; A61K 31/405; A61K 31/4172; A61K 35/20; A61K 38/17; C07K 16/00

(52) U.S. Cl. CPC A61K 39/395; A23L 1/3051; A23L 1/3056; A61K 38/1719; A61K 38/39; A61K 38/42; A61K 38/1722; C07K 16/00; A61K 38/1709; A61K 31/198; A61K 31/405; A61K 31/4172; A61K 35/20; A61K 9/0053

(57) ABSTRACT

The disclosure relates to supplement formulations comprising amino acids in ratios that are protein-specific. Described herein is a protein-specific supplement formulation comprising amino acids in the ratio present in the protein's polypeptide sequence. Further described herein is a protein-specific supplement formulation comprising: a base protein and essential amino acids; wherein the relative amounts of amino acids in the formulation match the relative amounts of amino acids naturally occurring in the protein.

### CROSS-REFERENCE

This application claims the benefit of U.S. provisional application Ser. No. 61/891,868, filed Oct. 16, 2013, which is incorporated by reference in its entirety.

### BACKGROUND

Amino acids are the building blocks of proteins and are used by organisms in the biosynthesis of proteins.

### SUMMARY OF THE INVENTION

Described herein is a protein-specific supplement formulation comprising amino acids in the ratio present in the protein's polypeptide sequence. Further described herein is a protein-specific supplement formulation comprising: a base protein and essential amino acids; wherein the relative amounts of amino acids in the formulation match the relative amounts of amino acids naturally occurring in the protein.

Also described is a protein-specific supplement formulation comprising essential amino acids in the ratio present in the protein's polypeptide sequence.

Also described is a protein-specific supplement formulation comprising: a base protein and essential amino acids; wherein the relative amounts of amino acids in the formulation match the relative amounts of amino acids naturally occurring in the protein.

Also described is a protein-specific supplement formulation, wherein essential amino acids are combined with a base protein to match the amounts of essential amino acids naturally occurring in a specific protein.

Also described is a protein-specific supplement formulation comprising: a base protein; an essential amino acid; a post-translational modification selected from the group consisting of myristoylation, palmitoylation, attachment of palmitate, isoprenylation or prenylation, farnesylation, geranylgeranylation, glypiation, glycosylphosphatidylinositol (GPI) anchor formation via an amide bond to C-terminal tail, lipoylation, flavin moiety (FMN or FAD) may be covalently attached, heme C attachment via thioether bonds with cysteine, phosphopantetheinylation, retinylidene Schiff base formation, diphthamide formation, ethanolamine phosphoglycerol attachment, hypusine formation, acylation, acetylation, deacetylation, formylation, alkylation, the addition of an alkyl group, e.g. methyl, ethyl, methylation, demethylation, amide bond formation, amidation at C-terminus, amino acid addition, arginylation, polyglutamylation, polyglycylation, butyrylation, gamma-carboxylation, glycosylation, glycation, polysialylation, malonylation, hydroxylation, iodination, nucleotide addition, oxidation, phosphate ester (O-linked) or phosphoramidate (N-linked) formation, phosphorylation, adenylylation, propionylation, pyroglutamate formation, S-glutathionylation, S-nitrosylation, succinylation, sulfation, selenoylation, biotinylation, pegylation, ISGylation, SUMOylation, ubiquitination, Neddylation, Pupylation, citrullination, deimination, deamidation, elimination, carbamylation, disulfide bridge formation, proteoltyic cleavage, and racemization; wherein the relative amounts of amino acids in the formulation match the relative amounts of amino acids naturally occurring the protein.

In some embodiments, the protein is actin. In some embodiments, the protein is myosin. In some embodiments, the protein is found in cardiac muscle. In some embodiments, the protein is found in skeletal muscle. In some embodiments, the protein is found in smooth muscle. In some embodiments, the protein is found in fast twitch muscle. In some embodiments, the protein is found in slow twitch muscle.

In some embodiments, the protein is found in blood. In some embodiments, the protein is found in tendons. In some embodiments, the protein is found in cartilage. In some embodiments, the protein is collagen. In some embodiments, the protein is hemoglobin. In some embodiments, the protein is found in haptoglobin. In some embodiments, the protein is immunoglobin. In some embodiments, the protein is found in lubricin. In some embodiments, the protein is aggrecan. In some embodiments, the protein is actin, aggrecan, androgens, arp2/3, c1-inhibitor, c3-convertase, cadherin, calcium channels, CFTR, c-myc, collagen, complement proteins, coronin, c-reactive protein, dystrophin, elastin, ependymin, epidermal growth factor, estrogen receptor, factor VIII, factor XIII, ferritin, fibrin, fibroblast growth factor, fibronextin, FOXP2, FOXP3, f-spondin, GABAa receptors, glucose transporter, glycophorin D, g-protein-coupled receptor, growth factors, haptoglobin, hemoglobin, histones, immunoglobins, insulin, insulin-like growth factor, integrin, keratin, ligan-gated ion channels, lubricin, major histocompatibility antigens, MyoD, myosin, NCAM, nebulin, nicotinic acetylcholine receptor, nucleosome, oxytocin, P53, peptide hormones, pikachurin, potassium channels, progesterones, protamines, protein C, protein S, protein Z, protein Z-related protease inhibitor, rhodopsin, ribonucleoprotein, scramblase, selectin, serum albumin, serum Amyloid P component, sex steroids, signal recognition particle, sodium channels, spectrin, spiceosome, steroid hormones, t-cell receptor, tau, thrombin, titin, tropomyosin, tubulin, vascular endothelial growth factor, voltage-gated ion channels, or Van Wllebrand factor.

Also disclosed herein is a formulation for actin development, comprising L-histidine, L-methionine, L-isoleucine, L-leucine, L-lysine, L-phenylalanine, L-threonine, L-tryptophan, and L-valine in ratios present in the actin's polypeptide sequence. In some cases, the formulation further comprises a base protein. In some embodiments, the base protein is whey protein, milk protein, egg-white protein, hemp seed protein, soy protein, rice protein, pea protein, lactalbumin, or a combination thereof. In some embodiments, the formulation comprises the following percentages of amino acids: 18.2% of L-isoleucine; 15.8% of L-leucine; 11.5% of L-lysine; 7.3% of L-phenylalanine; 16.4% of L-threonine; 2.4% of L-tryptophan; 12.7% of L-valine; 5.5% of L-histidine; and 10.3% of L-methionine

In some embodiments, the amino acids are free amino acids. In some embodiments, the formulation further comprises vitamins, electrolytes, minerals, antioxidants, digestive enzymes, coenzymes, or a combination thereof. In some embodiments, the formulation further comprises flavoring agents, thickeners, fillers, preservatives, fiber, fatty acids, binding agents, or a combination thereof.

Described herein is a formulation for hemoglobin development, comprising L-leucine, L-lysine, L-methionine, L-threonine, L-tryptophan, L-valine, L-histidine, and L-phenylalanine in ratios present in hemoglobin's polypeptide sequence. In some embodiments, the formulation further comprises a base protein. In some embodiments, the base protein is whey protein, milk protein, egg-white protein, hemp seed protein, soy protein, rice protein, pea protein, lactalbumin, or a combination thereof. In some embodiments, the formulation comprises the following percentages of amino acids: 24.5% of L-leucine; 15.1% of L-lysine; 3.8% of L-methionine; 11.3% of L-threonine; 3.8% of L-tryptophan;

20.8% of L-valine; 9.4% of L-histidine; and 11.3% of L-phenylalanine. In some embodiments, the amino acids are free amino acids. In some embodiments, the formulation further comprises vitamins, electrolytes, minerals, antioxidants, digestive enzymes, coenzymes, or a combination thereof. In some embodiments, the formulation further comprises flavoring agents, thickeners, fillers, preservatives, fiber, fatty acids, binding agents, or a combination thereof.

Described herein is a formulation for collagen development, comprising L-isoleucine, L-leucine, L-lysine, L-methionine, L-threonine, L-tryptophan, L-valine, L-histidine, and L-phenylalanine in ratios present in collagen's polypeptide sequence. In some embodiments, the formulation further comprises a base protein. In some embodiments, the base protein is whey protein, milk protein, egg-white protein, hemp seed protein, soy protein, rice protein, pea protein, lactalbumin, or a combination thereof. In some embodiments, the formulation comprises the following percentages of amino acids: 8.7% of L-isoleucine; 17.4% of L-leucine; 20.7% of L-lysine; 4.7% of L-methionine; 16.3% of L-threonine; 2.2% of L-tryptophan; 17.0% of L-valine; 9.8% of L-phenylalanine; and 3.3% of L-histidine.

In some embodiments, the amino acids are free amino acids. In some embodiments, the formulation further comprises vitamins, electrolytes, minerals, antioxidants, digestive enzymes, coenzymes, or a combination thereof. In some embodiments, the formulation further comprises flavoring agents, thickeners, fillers, preservatives, fiber, fatty acids, binding agents, or a combination thereof.

Described herein is a formulation for haptoglobin development, comprising L-isoleucine, L-leucine, L-lysine, L-methionine, L-phenylalanine, L-threonine, L-tryptophan, L-histidine, and L-valine in ratios present in haptoglobin's polypeptide sequence. In some embodiments, the formulation further comprises a base protein. In some embodiments, the base protein is whey protein, milk protein, egg-white protein, hemp seed protein, soy protein, rice protein, pea protein, lactalbumin, or a combination thereof. In some embodiments, the formulation comprises the following percentages of amino acids: 10.2% of L-isoleucine; 17.6% of L-leucine; 19.9% of L-lysine; 2.8% of L-methionine; 4.5% of L-phenylalanine; 12.5% of L-threonine; 4.5% of L-tryptophan; 7.4% of L-histidine; and 20.5% of L-valine.

In some embodiments, the amino acids are free amino acids. In some embodiments, the formulation further comprises vitamins, electrolytes, minerals, antioxidants, digestive enzymes, coenzymes, or a combination thereof. In some embodiments, the formulation further comprises flavoring agents, thickeners, fillers, preservatives, fiber, fatty acids, binding agents, or a combination thereof.

Described herein is a formulation for lubricin development, comprising L-isoleucine, L-leucine, L-lysine, L-methionine, L-phenylalanine, L-tryptophan, L-valine, L-histidine, and L-threonine in ratios present in lubricin's polypeptide sequence. In some embodiments, the formulation further comprises a base protein. In some embodiments, the base protein is whey protein, milk protein, egg-white protein, hemp seed protein, soy protein, rice protein, pea protein, lactalbumin, or a combination thereof. In some embodiments, the formulation comprises the following percentages of amino acids: 5.5% of L-isoleucine; 7.7% of L-leucine; 25.3% of L-lysine; 2.2% of L-methionine; 3.6% of L-phenylalanine; 1.2% of L-tryptophan; 6.3% of L-valine; 1.2% of L-histidine; and 47.0% of L-threonine.

In some embodiments, the amino acids are free amino acids. In some embodiments, the formulation further comprises vitamins, electrolytes, minerals, antioxidants, digestive enzymes, coenzymes, or a combination thereof. In some embodiments, the formulation further comprises flavoring agents, thickeners, fillers, preservatives, fiber, fatty acids, binding agents, or a combination thereof

Described herein is a formulation for aggrecan development, comprising L-isoleucine, L-leucine, L-lysine, L-methionine, L-threonine, L-tryptophan, L-valine, L-histidine, and L-phenylalanine in ratios present in aggrecan's polypeptide sequence. In some embodiments, the formulation further comprises a base protein. In some formulations, the base protein is whey protein, milk protein, egg-white protein, hemp seed protein, soy protein, rice protein, pea protein, lactalbumin, or a combination thereof. In some embodiments, the formulation comprises the following percentages of amino acids: 9.2% of L-isoleucine; 19.4% of L-leucine; 5.1% of L-lysine; 3.3% of L-methionine; 22.3% of L-threonine; 3.7% of L-tryptophan; 20.9% of L-valine; 5.1% of L-histidine; and 11.4% of L-phenylalanine.

In some embodiments, the amino acids are free amino acids. In some embodiments, the formulation further comprises vitamins, electrolytes, minerals, antioxidants, digestive enzymes, coenzymes, or a combination thereof. In some embodiments, the formulation further comprises flavoring agents, thickeners, fillers, preservatives, fiber, fatty acids, binding agents, or a combination thereof.

Described herein is a formulation for immunoglobin development, comprising L-isoleucine, L-leucine, L-lysine, L-methionine, L-threonine, L-tryptophan, L-valine, L-histidine, and L-phenylalanine in ratios present in immunoglobin's polypeptide sequence. In some embodiments, the formulation further comprises a base protein. In some embodiments, the base protein is whey protein, milk protein, egg-white protein, hemp seed protein, soy protein, rice protein, pea protein, lactalbumin, or a combination thereof. In some embodiments, the formulation comprises the following residue percentages of amino acids: 10.1% of L-isoleucine; 23.2% of L-leucine; 7.1% of L-lysine; 6.1% of L-methionine; 19.2% of L-threonine; 2.0% of L-tryptophan; 19.7% of L-valine; 8.6% L-phenylalanine; and 4.0% of L-histidine.

In some embodiments, the amino acids are free amino acids. In some embodiments, the formulation further comprises vitamins, electrolytes, minerals, antioxidants, digestive enzymes, coenzymes, or a combination thereof. In some embodiments, the formulation further comprises flavoring agents, thickeners, fillers, preservatives, fiber, fatty acids, binding agents, or a combination thereof.

Described herein is a formulation for myosin development, comprising L-isoleucine, L-leucine, L-lysine, L-methionine, L-phenylalanine, L-threonine, L-tryptophan, L-valine, and L-histidine in ratios present in Myosin's polypeptide sequence. In some embodiments, the formulation further comprises a base protein. In some embodiments, the base protein is whey protein, milk protein, egg-white protein, hemp seed protein, soy protein, rice protein, pea protein, lactalbumin, or a combination thereof. In some embodiments, the formulation comprises the following percentages of amino acids: 10.8% of L-isoleucine; 24.1% of L-leucine; 25.0% of L-lysine; 5.2% of L-methionine; 6.4% of L-phenylalanine; 11.7% of L-threonine; 1.2% of L-tryptophan; 10.9% of L-valine; and 4.5% of L-histidine.

In some embodiments, the amino acids are free amino acids. In some embodiments, the formulation further comprises vitamins, electrolytes, minerals, antioxidants, digestive enzymes, coenzymes, or a combination thereof. In some embodiments, the formulation further comprises flavoring agents, thickeners, fillers, preservatives, fiber, fatty acids, binding agents, or a combination thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an illustrative example of how proteins are synthesized in the human body.

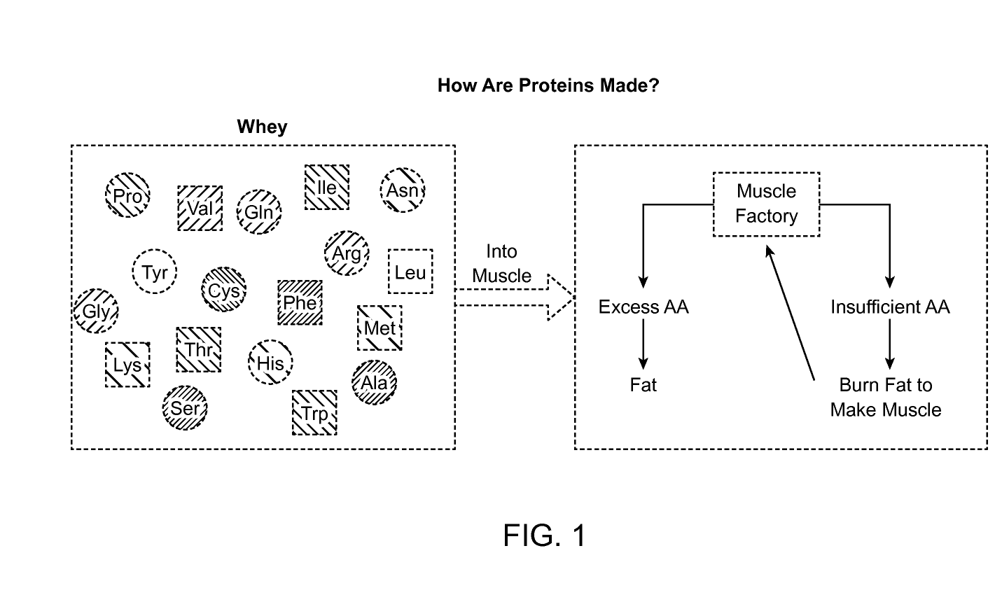

FIG. 2 shows an illustrative example of the rate limiting step of protein biosynthesis.

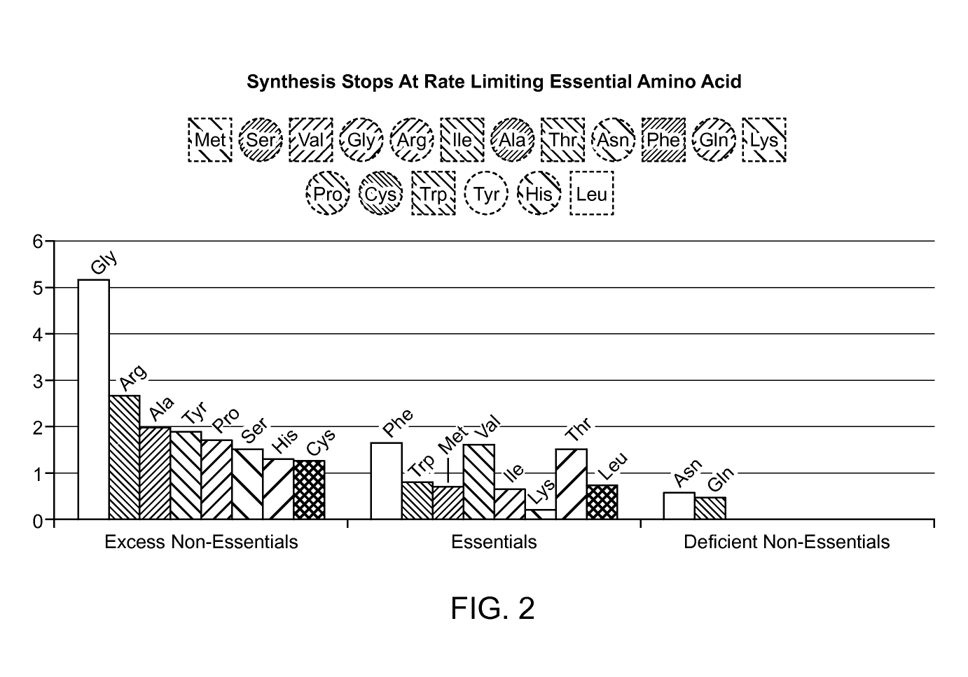

FIG. 3 shows an illustrative example of the ratio of amino acids in whey protein.

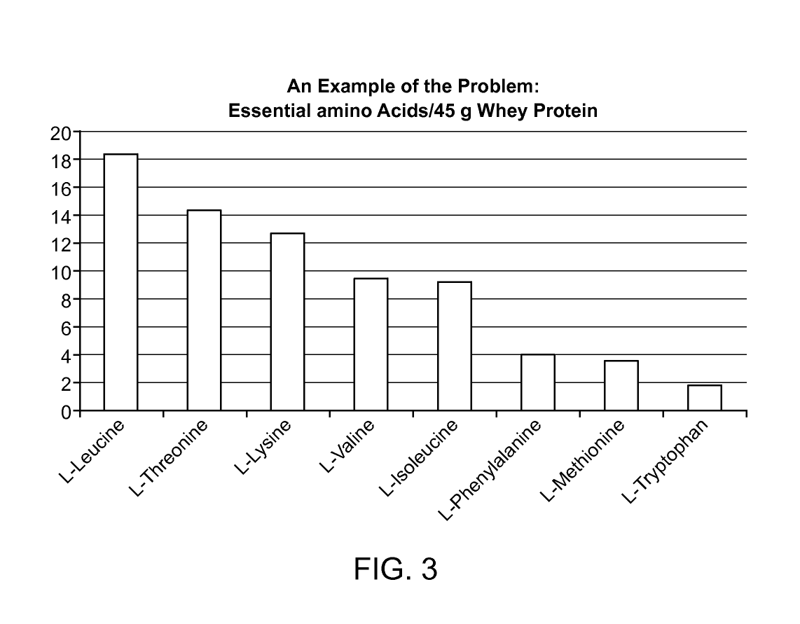

FIG. 4 shows an illustrative example of the ratio of essential amino acids in skeletal muscle.

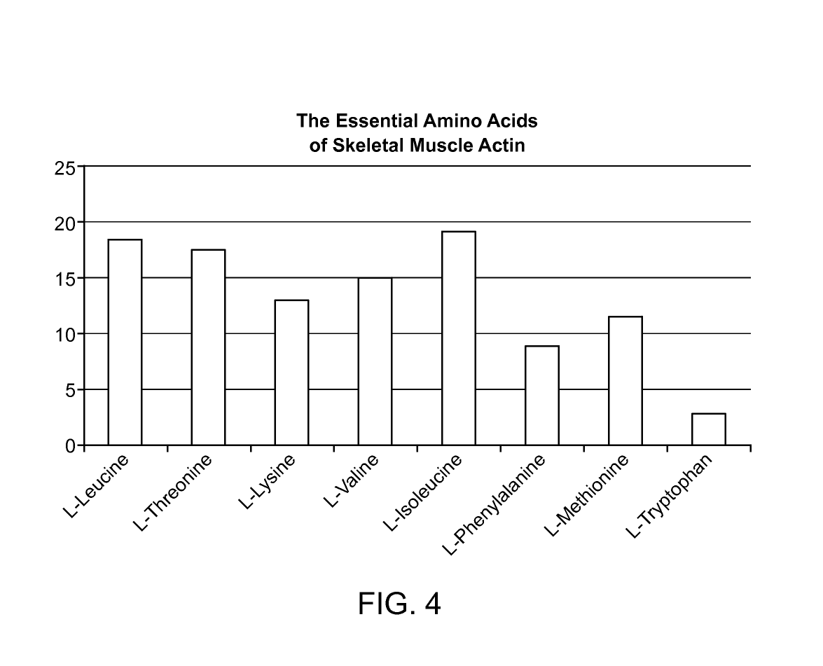

FIG. 5 shows an illustrative example of the conversion of whey protein to muscle protein.

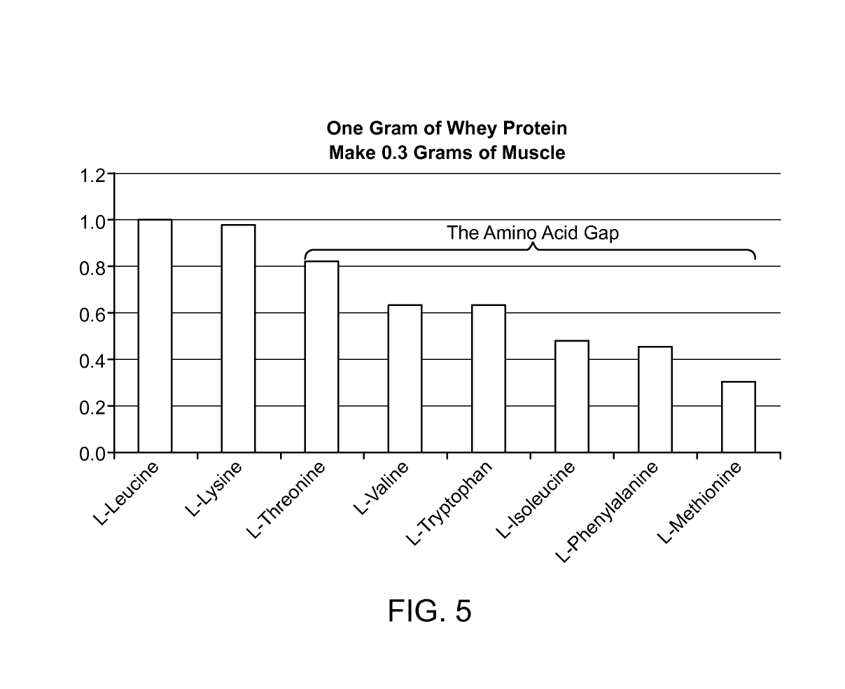

FIG. 6 shows an illustrative example of tissue-specific protein supplements.

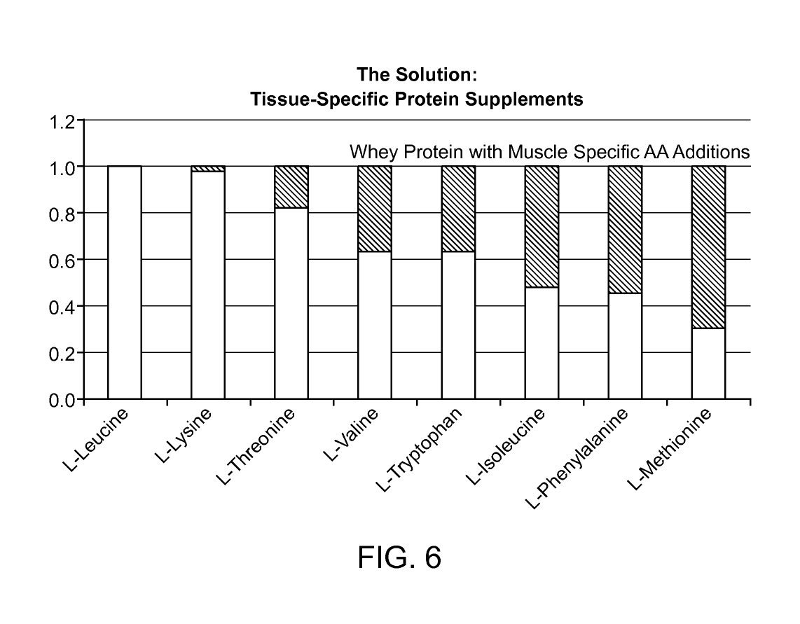

### DETAILED DESCRIPTION OF THE INVENTION

Protein formulations are useful for the development of muscle, tissue, and peptides in humans. Amino acids are the building blocks of protein and are used by organisms in the biosynthesis of a variety of proteins, including, but not limited to, structural proteins, enzymes, some hormones and neurotransmitters. Currently available protein formulations are primarily composed of an inexpensive protein source, e.g., milk protein, and lack the proper ratios of essential amino acids required for the biosynthesis of specific peptides in humans. Described herein are protein formulations that comprise the correct residue percentages of essential amino acids required for the biosynthesis of specific peptides in humans.

“polypeptide” and “peptide,” as used herein, are used interchangeably and include, but are not limited to, two or more amino acids bonded together via an amide bond.

“Amino acid,” as used herein, includes, but is not limited to, amino acids that are naturally occurring, synthetic, essential, non-essential, standard, non-standard, and non-naturally occurring.

### Proteins Targeted by the Formulations

The formulations described herein supplement a naturally-occurring protein by matching the amounts of essential amino acids in the formulation to a targeted protein.

The formulations described herein target a single protein or more than one protein. In some embodiments, the formulations described herein target a single protein found in a muscle or tissue. In some embodiments, a formulation described herein targets more than one protein found in a particular muscle or tissue. In some embodiments, a formulation targets more than one protein in more than one tissue or muscle.

In some embodiments, a formulation described herein is specific to a particular isoform of a protein. In some embodiments, a formulation described herein targets more than one isoform of a protein.

In some embodiments, a formulation described herein is specific to one or more proteins encoded by a particular gene. In some embodiments, a formulation described herein is specific to a group of proteins encoded by a particular gene.

### Base Protein Sources

In some embodiments, the protein formulations described herein comprise a base protein source and supplemental amino acids. In some embodiments, the base protein is derived from a vegetable source. In some embodiments, the base protein is derived from a milk source. In some embodiments, the base protein is derived from a grain or carbohydrate source. In some embodiments, the base protein source is optionally selected from whey protein, milk protein or casein, egg-white, protein, hemp seed, soy protein, rice protein, pea protein, lactalbumin, or a combination thereof.

In some embodiments, the base protein is in concentrate form. In some embodiments, the base protein is a protein isolate. In some embodiments, formulations described herein lack a base protein.

In some embodiments, the formulations described herein comprise whey protein isolate. In some embodiments, the formulations comprise whey protein concentrate. In some embodiments, the formulations comprise a combination of whey protein isolate and whey protein concentrate.

In some embodiments, the base protein is further processed prior to its use in the formulation.

In some embodiments, the composition of the base protein used is analyzed for essential amino acid composition prior to its use in the formulation. In some embodiments, the base protein is purified prior to use.

### Amino Acid Sources

Amino acids and their derivatives are commercially available from a wide range of chemical suppliers. In some embodiments, amino acids are in a chelated form. In some embodiments, amino acids are present in their free form. In some embodiments, amino acid derivatives, e.g., ester derivatives are used. In some embodiments, amino acids are derived from a base protein. Amino acids include, but are not limited to, essential, non-essential, standard, non-standard, naturally occurring, non-naturally occurring, and synthetic amino acids.

In some embodiments, the amino acid is provided as any active form of an amino acid. In some embodiments, the amino acid is derived from a compound that when administered to the body is converted to the amino acid, which then appears in the blood, plasma or serum and is available for uptake into muscles, tissues, and cells.

### Ratios of Amino Acids

Described herein are formulations comprising a ratio of essential amino acid that is roughly equivalent to the ratio of essential amino acid residues in a target protein. The ratios are determined from the sequence of the targeted protein. In some embodiments, the formulations comprise amino acids that are non-essential, synthetic, or non-naturally occuring. In some embodiments, the formulations comprise ratios of amino acids that vary from the ratio of amino acids in the targeted protein.

In some embodiments, the percentage of essential amino acid residues required for the biosynthesis of a specific protein is known to those skilled in the art. In some embodiments, the ratio of essential amino acids required for the formulations described herein are determined empirically via analysis of the sequences of particular proteins using methods known to those skilled in the art.

In some embodiments, the ratio of essential amino acids in the formulations described herein is altered to the individual subject, taking into account factors such as age, weight, and healthiness.

### Ratio of Essential Amino Acid to Base Protein

In some embodiments, the formulation comprises essential amino acids in the ratio present in a target protein's polypeptide sequence. In some embodiments, the essential amino acids are derived from the base protein, separate essential amino acid sources, or a combination thereof. In some embodiments, the relative amounts of essential amino acids in the formulation match the relative amounts of essential amino acids naturally occurring in the protein. In some embodiments, the essential amino acids in the formulation are provided by whey protein isolate, hydrolyzed whey protein, whey protein concentrate, individual essential amino acids, L-form essential amino acids, or a combination thereof.

In some embodiments, the base protein is analyzed prior to use to determine the amount of essential amino acids present. In some embodiments, the ratio of the essential amino acids to the base protein is determined as a weight/weight ratio. In some embodiments, the ratio refers to a percentage of essential amino acids.

The weight/weight ratio of the base protein to the essential amino acid is dependent upon the base protein. Different base proteins comprise different amounts of essential amino acids; therefore, the ratio of base protein to the essential amino acid varies with each base protein. The process by which base proteins are processed alters the amount of essential amino acid in the base protein. Variations in the amount of essential amino acids in different base protein sources are taken into account in determining the weight/weight ratios.

### w/w Ratio of Base Protein:L-Isoleucine

In some embodiments of the formulation comprising base protein and essential amino acids, the weight/weight ratio of the base protein to L-isoleucine is between about 5:1 and 20:1. In some embodiments, the weight/weight (w/w) ratio of the base protein to the essential amino acid is between about 7:1 and 17:1.

In some embodiments of the formulation for actin development, the w/w ratio of the base protein to L-isoleucine is between about 13:1 and 19:1. In some embodiments of the formulation for skeletal muscle development, the w/w ratio of the base protein to L-isoleucine is between about 15:1 to about 17:1. In some embodiments of the formulation for skeletal muscle development, the w/w ratio of the base protein to L-isoleucine is about 16:1. In another embodiment, the formulation for skeletal muscle development comprises a w/w ratio of whey protein to L-isoleucine of about 16:1.

In some embodiments of the formulation for collagen development, the w/w ratio of the base protein to L-isoleucine is between about 9.5:1 and about 15.5:1. In some embodiments of the formulation for collagen development, the w/w ratio of the base protein to L-isoleucine is between about 11.5:1 and about 13.5:1. In some embodiments of the formulation for collagen development, the w/w ratio of the base protein to L-isoleucine is about 12.5:1. In another embodiment of the formulation for collagen development, the w/w ratio of whey protein to L-isoleucine is about 12.5:1.

In some embodiments of the formulation for the development of a haptoglobin protein, the w/w ratio of the base protein to L-isoleucine is between about 9.7:1 and about 15.7:1; between about 11.7:1 and about 13.7:1; or between about 12:1 and 13:1. In another embodiment of the formulation for the development of a haptoglobin protein, the w/w ratio of the base protein to L-isoleucine is about 12.7:1. In some embodiments of the formulation for development of a haptoglobin protein, the w/w ratio of the whey protein to L-isoleucine is about 12.7:1.

In some embodiments of the formulation for the development of an immunoglobin, the w/w ratio of the base protein to L-isoleucine is between about 10:1 and about 16:1; between about 12:1 and about 14:1; or between about 13:1 and 13.5:1. In some embodiments of the formulation for development of an immunoglobin protein, the w/w ratio of the base protein to L-isoleucine is about 13.25:1. In another embodiment of the formulation for development of an immunoglobin protein, the w/w ratio of whey protein to L-isoleucine is about 13.25:1.

In some embodiments of the formulation for the development of lubricin, the w/w ratio of the base protein to L-isoleucine is between about 5:1 and about 11:1; between about 7:1 and about 9:1; and between about 7.5:1 and about 8.5:1. In some embodiments of the formulation for development of lubricin, the w/w ratio of the base protein to L-isoleucine is about 8:1. In another embodiment of the formulation for the development of lubricin, the w/w ratio of whey protein to L-isoleucine is about 8:1.

### w/w Ratio of Base Protein:L-Leucine

In some embodiments of the formulation comprising base protein and essential amino acids, the weight/weight ratio of the base protein to L-leucine is between about 1:1 and 12:1; between about 3:1 and about 10:1; or between about 4:1 and about 9:1.

In some embodiments of the formulation for skeletal muscle development, the w/w ratio of the base protein to L-leucine is between about 2:1 and 8:1; between about 4:1 and about 6:1; or between about 4.5:1 and about 5.5:1. In some embodiments of the formulation for skeletal muscle development, the w/w ratio of the base protein to L-leucine is about 5:1. In some embodiments of the formulation for skeletal muscle development, the w/w ratio of the base protein to L-leucine is about 5.2:1. In another embodiment, the formulation for skeletal muscle development comprises a w/w ratio of whey protein to L-leucine of about 5:1.

In some embodiments of the formulation for hemoglobin development, the w/w ratio of the base protein to L-leucine is between about 6:1 and 12:1; between about 8:1 and about 10:1; or between about 8.5:1 and about 9.5:1. In some embodiments of the formulation for hemoglobin development, the w/w ratio of the base protein to L-leucine is about 5:1. In some embodiments of the formulation for hemoglobin development, the w/w ratio of the base protein to L-leucine is about 9:1. In another embodiment, the formulation for hemoglobin development comprises a w/w ratio of whey protein to L-leucine of about 9:1.

In some embodiments of the formulation for collagen development, the w/w ratio of the base protein to L-leucine is between about 4:1 and about 10:1; between about 6:1 and about 8:1; or between about 7:1 and about 8:1. In some embodiments of the formulation for collagen development, the w/w ratio of the base protein to L-leucine is about 7.5:1. In another embodiment of the formulation for collagen development, the w/w ratio of whey protein to L-leucine is about 7.5:1.

In some embodiments of the formulation for haptoglobin development, the w/w ratio of the base protein to L-leucine is between about 3.5:1 and about 9.5:1; between about 5.5:1 and about 7.6:1; or between about 6:1 and about 7:1. In some embodiments of the formulation for haptoglobin development, the w/w ratio of the base protein to L-leucine is about 6.5:1. In another embodiment of the formulation of haptoglobin development, the w/w ratio of whey protein to L-leucine is about 6.5:1.

In some embodiments of the formulation for immunoglobin development, w/w ratio of the base protein to L-leucine is between about 6:1 and about 12:1; between about 8:1 and 10:1; or between about 8.5:1 and about 9.5:1. In some embodiments of the formulation for immunoglobin development, the w/w ratio of the base protein to L-leucine is about 9:1. In another embodiment of the formulation for immunoglobin development, the w/w ratio of the base protein to L-leucine is about 9.2:1.

In some embodiments of the formulation for lubricin development, the w/w ratio of the base protein to L-leucine is between about 1:1 and about 7:1; between about 3:1 and about 5:1; or between about 3.5:1 and about 4.5:1. In some embodiments of the formulation for lubricin development, the w/w ratio of the base protein to L-leucine is about 4:1. In another embodiment of the formulation for lubricin development, the w/w ratio of the whey protein to L-leucine is about 4.2:1.

In some embodiments of the formulation for aggrecan development, the w/w ratio of the base protein to L-leucine is between about 4:1 and about 10:1; between about 6:1 and about 8:1; or between about 6.5:1 and about 7.5:1. In some embodiments of the formulation for aggrecan development, the w/w ratio of the base protein to L-leucine is about 7:1. In another embodiment of the formulation for aggrecan development, the w/w ratio of whey protein to L-leucine is about 6.9:1.

### w/w Ratio of Base Protein:L-Lysine

In some embodiments of the formulation comprising base protein and essential amino acids, the weight/weight ratio of the base protein to L-lysine is between about 2:1 and about 17:1; between about 4:1 and about 15:1; or between about 5:1 and about 14:1.

In some embodiments of the formulation for skeletal muscle development, the w/w ratio of the base protein to L-lysine is between about 2:1 and 10:1; between about 4:1 and about 8:1; or between about 5:1 and about 7:1. In some embodiments of the formulation for skeletal muscle development, the w/w ratio of the base protein to L-lysine is about 6:1. In some embodiments of the formulation for skeletal muscle development, the w/w ratio of the base protein to L-lysine is about 5.7:1. In another embodiment, the formulation for skeletal muscle development comprises a w/w ratio of whey protein to L-lysine of about 5.7:1.

In some embodiments of the formulation for hemoglobin development, the w/w ratio of the base protein to L-lysine is between about 4:1 and 11:1; between about 6:1 and about 9:1; or between about 7:1 and about 8:1. In some embodiments of the formulation for hemoglobin development, the w/w ratio of the base protein to L-lysine is about 7.5:1. In some embodiments of the formulation for hemoglobin development, the w/w ratio of the base protein to L-lysine is about 7.5:1. In another embodiment, the formulation for hemoglobin development comprises a w/w ratio of whey protein to L-lysine of about 7.5:1.

In some embodiments of the formulation for collagen development, the w/w ratio of the base protein to L-lysine is between about 10:1 and about 17:1; between about 12:1 and about 15:1; or between about 13:1 and about 14:1. In some embodiments of the formulation for collagen development, the w/w ratio of the base protein to L-lysine is about 13.5:1. In another embodiment of the formulation for collagen development, the w/w ratio of whey protein to L-lysine is about 13.5:1.

In some embodiments of the formulation for haptoglobin development, the w/w ratio of the base protein to L-lysine is between about 7:1 and about 13:1; between about 9:1 and about 11:1; or between about 9.5:1 and about 10.5:1. In some embodiments of the formulation for haptoglobin development, the w/w ratio of the base protein to L-lysine is about 10:1. In another embodiment of the formulation of haptoglobin development, the w/w ratio of whey protein to L-lysine is about 10:1.

In some embodiments of the formulation for immunoglobin development, w/w ratio of the base protein to L-lysine is between about 2:1 and about 9:1; between about 4:1 and 7:1; or between about 5:1 and about 6:1. In some embodiments of the formulation for immunoglobin development, the w/w ratio of the base protein to L-lysine is about 5.5:1. In another embodiment of the formulation for immunoglobin development, the w/w ratio of the base protein to L-lysine is about 5.5:1.

In some embodiments of the formulation for lubricin development, the w/w ratio of the base protein to L-lysine is between about 4:1 and about 10:1; between about 6:1 and about 8:1; or between about 6.5:1 and about 7.5:1. In some embodiments of the formulation for lubricin development, the w/w ratio of the base protein to L-lysine is about 7:1. In another embodiment of the formulation for lubricin development, the w/w ratio of the whey protein to L-lysine is about 7:1.

In some embodiments of the formulation for aggrecan development, the w/w ratio of the base protein to L-lysine is between about 2:1 and about 8:1; between about 4:1 and about 6:1; or between about 4.5:1 and about 5.5:1. In some embodiments of the formulation for aggrecan development, the w/w ratio of the base protein to L-lysine is about 5:1. In another embodiment of the formulation for aggrecan development, the w/w ratio of whey protein to L-lysine is about 5:1.

### w/w Ratio of Base Protein:L-Methionine

In some embodiments of the formulation comprising base protein and essential amino acids, the weight/weight ratio of the base protein to L-methionine is between about 20:1 and about 52:1; between about 23:1 and about 49:1; or between about 25:1 and about 47:1.

In some embodiments of the formulation for hemoglobin development, the w/w ratio of the base protein to L-methionine is between about 43:1 and about 49:1; between about 45:1 and about 47:1; or between about 45.5:1 and about 46.5:1. In some embodiments of the formulation for hemoglobin development, the w/w ratio of the base protein to L-methionine is about 46:1. In some embodiments of the formulation for hemoglobin development, the w/w ratio of the base protein to L-methionine is about 46:1. In another embodiment, the formulation for hemoglobin development comprises a w/w ratio of whey protein to L-methionine is about 46:1.

In some embodiments of the formulation for collagen development, the w/w ratio of the base protein to L-methionine is between about 96:1 and about 102:1; between about 98:1 and about 100:1; or between about 98.5:1 and about 99.5:1. In some embodiments of the formulation for collagen development, the w/w ratio of the base protein to L-methionine is about 99:1. In another embodiment of the formulation for collagen development, the w/w ratio of whey protein to L-methionine is about 99:1.

In some embodiments of the formulation for haptoglobin development, the w/w ratio of the base protein to L-methionine is between about 33:1 and about 39:1; between about 35:1 and about 37:1; or between about 35.5:1 and about 36.5:1. In some embodiments of the formulation for haptoglobin development, the w/w ratio of the base protein to L-methionine is about 36:1. In another embodiment of the formulation of haptoglobin development, the w/w ratio of whey protein to L-methionine is about 36:1.

In some embodiments of the formulation for immunoglobin development, w/w ratio of the base protein to L-methionine is between about 213:1 and about 219:1; between about 215:1 and about 217:1; or between about 215.5:1 and about 216.5:1. In some embodiments of the formulation for immunoglobin development, the w/w ratio of the base protein to L-methionine is about 216:1. In another embodiment of the formulation for immunoglobin development, the w/w ratio of the base protein to L-methionine is about 216:1.

In some embodiments of the formulation for lubricin development, the w/w ratio of the base protein to L-methionine is between about 23:1 and about 29:1; between about 25:1 and about 27:1; or between about 25.5:1 and about 26.5:1. In some embodiments of the formulation for lubricin development, the w/w ratio of the base protein to L-methionine is about 26:1. In another embodiment of the formulation for lubricin development, the w/w ratio of the whey protein to L-methionine is about 26:1.

In some embodiments of the formulation for aggrecan development, the w/w ratio of the base protein to L-methionine is between about 36:1 and about 42:1; between about 38:1 and about 40:1; or between about 38.5:1 and about 39.5:1. In some embodiments of the formulation for aggrecan development, the w/w ratio of the base protein to L-methionine is about 39:1. In another embodiment of the formulation for aggrecan development, the w/w ratio of whey protein to L-methionine is about 39:1.

### w/w Ratio to Base Protein:L-Phenylalanine

In some embodiments of the formulation comprising base protein and essential amino acids, the weight/weight ratio of the base protein to L-phenylalanine is between about 10:1 and about 80:1; between about 13:1 and about 77:1; between about 15:1 and about 75:1; or between about 17:1 and about 72:1.

In some embodiments of the formulation for skeletal muscle development, the w/w ratio of the base protein to L-phenylalanine is between about 23:1 and 30:1; between about 25:1 and about 28:1; or between about 26:1 and about 27:1. In some embodiments of the formulation for skeletal muscle development, the w/w ratio of the base protein to L-phenylalanine is about 26:1. In some embodiments of the formulation for skeletal muscle development, the w/w ratio of the base protein to L-phenylalanine is about 26.4:1. In another embodiment, the formulation for skeletal muscle development comprises a w/w ratio of whey protein to L-phenylalanine of about 26.4:1.

In some embodiments of the formulation for haptoglobin development, the w/w ratio of the base protein to L-phenylalanine is between about 21:1 and about 28:1; between about 23:1 and about 26:1; or between about 24:1 and about 25:1. In some embodiments of the formulation for haptoglobin development, the w/w ratio of the base protein to L-phenylalanine is about 24.6:1. In another embodiment of the formulation of haptoglobin development, the w/w ratio of whey protein to L-phenylalanine is about 24.6:1.

In some embodiments of the formulation for immunoglobin development, w/w ratio of the base protein to L-phenylalanine is between about 68:1 and about 75:1; between about 70:1 and 73:1; or between about 71:1 and about 72:1. In some embodiments of the formulation for immunoglobin development, the w/w ratio of the base protein to L-phenylalanine is about 71.4:1. In another embodiment of the formulation for immunoglobin development, the w/w ratio of the base protein to L-phenylalanine is about 71.4:1.

In some embodiments of the formulation for lubricin development, the w/w ratio of the base protein to L-phenylalanine is between about 14:1 and about 20:1; between about 16:1 and about 18:1; or between about 6.5:1 and about 17.5:1. In some embodiments of the formulation for lubricin development, the w/w ratio of the base protein to L-phenylalanine is about 17:1. In another embodiment of the formulation for lubricin development, the w/w ratio of the whey protein to L-phenylalanine is about 17.2:1.

### w/w Ratio to Base Protein:L-Threonine

In some embodiments of the formulation comprising base protein and essential amino acids, the weight/weight ratio of the base protein to L-threonine is between about 3:1 and about 92:1; between about 6:1 and about 89:1; between about 9:1 and about 86:1; or between about 12:1 and about 83:1.

In some embodiments of the formulation for skeletal muscle development, the w/w ratio of the base protein to L-threonine is between about 9:1 and 16:1; between about 11:1 and about 14:1; or between about 12:1 and about 13:1. In some embodiments of the formulation for skeletal muscle development, the w/w ratio of the base protein to L-threonine is about 12.8:1. In some embodiments of the formulation for skeletal muscle development, the w/w ratio of the base protein to L-threonine is about 12.8:1. In another embodiment, the formulation for skeletal muscle development comprises a w/w ratio of whey protein to L-threonine of about 12.8:1.

In some embodiments of the formulation for hemoglobin development, the w/w ratio of the base protein to L-threonine is between about 9:1 and 16:1; between about 11:1 and about 14:1; or between about 12:1 and about 13:1. In some embodiments of the formulation for hemoglobin development, the w/w ratio of the base protein to L-threonine is about 12.4:1. In another embodiment, the formulation for hemoglobin development comprises a w/w ratio of whey protein to L-threonine of about 12.4:1.

In some embodiments of the formulation for collagen development, the w/w ratio of the base protein to L-threonine is between about 30:1 and about 37:1; between about 32:1 and about 35:1; or between about 33:1 and about 34:1. In some embodiments of the formulation for collagen development, the w/w ratio of the base protein to L-threonine is about 33.4:1. In another embodiment of the formulation for collagen development, the w/w ratio of whey protein to L-threonine is about 33.4:1.

In some embodiments of the formulation for haptoglobin development, the w/w ratio of the base protein to L-threonine is between about 11:1 and about 17:1; between about 13:1 and about 15:1; or between about 13.5:1 and about 14.5:1. In some embodiments of the formulation for haptoglobin development, the w/w ratio of the base protein to L-threonine is about 14:1. In another embodiment of the formulation of haptoglobin development, the w/w ratio of whey protein to L-threonine is about 14:1.

In some embodiments of the formulation for immunoglobin development, w/w ratio of the base protein to L-threonine is between about 38:1 and about 44:1; between about 40:1 and 42:1; or between about 40.5:1 and about 41.5:1. In some embodiments of the formulation for immunoglobin development, the w/w ratio of the base protein to L-threonine is about 41:1. In another embodiment of the formulation for immunoglobin development, the w/w ratio of the base protein to L-threonine is about 41:1.

In some embodiments of the formulation for aggrecan development, the w/w ratio of the base protein to L-threonine is between about 80:1 and about 86:1; between about 82:1 and about 84:1; or between about 82.5:1 and about 83.5:1. In some embodiments of the formulation for aggrecan development, the w/w ratio of the base protein to L-threonine is about 83:1. In another embodiment of the formulation for aggrecan development, the w/w ratio of whey protein to L-threonine is about 83:1.

### w/w Ratio of Base Protein:L-Tryptophan

In some embodiments of the formulation comprising base protein and essential amino acids, the weight/weight ratio of the base protein to L-tryptophan is between about 18:1 and about 67:1; between about 21:1 and about 64:1; or between about 24:1 and about 61:1.

In some embodiments of the formulation for skeletal muscle development, the w/w ratio of the base protein to L-tryptophan is between about 25:1 and about 32:1; between about 27:1 and about 30:1; or between about 28:1 and about 29:1. In some embodiments of the formulation for skeletal muscle development, the w/w ratio of the base protein to L-tryptophan is about 28.6:1. In another embodiment, the formulation for skeletal muscle development comprises a w/w ratio of whey protein to L-tryptophan of about 28.6:1.

In some embodiments of the formulation for hemoglobin development, the w/w ratio of the base protein to L-tryptophan is between about 40:1 and 48:1; between about 42:1 and about 46:1; or between about 43:1 and about 45:1. In some embodiments of the formulation for hemoglobin development, the w/w ratio of the base protein to L-tryptophan is about 44:1. In another embodiment, the formulation for hemoglobin development comprises a w/w ratio of whey protein to L-tryptophan of about 44.2:1.

In some embodiments of the formulation for collagen development, the w/w ratio of the base protein to L-tryptophan is between about 29:1 and about 37:1; between about 31:1 and about 35:1; or between about 32:1 and about 34:1. In some embodiments of the formulation for collagen development, the w/w ratio of the base protein to L-tryptophan is about 33:1. In another embodiment of the formulation for collagen development, the w/w ratio of whey protein to L-tryptophan is about 33:1.

In some embodiments of the formulation for haptoglobin development, the w/w ratio of the base protein to L-tryptophan is between about 57:1 and about 64:1; between about 59:1 and about 62:1; or between about 60:1 and about 61:1. In some embodiments of the formulation for haptoglobin development, the w/w ratio of the base protein to L-tryptophan is about 60.4:1. In another embodiment of the formulation of haptoglobin development, the w/w ratio of whey protein to L-tryptophan is about 60.4:1.

In some embodiments of the formulation for immunoglobin development, w/w ratio of the base protein to L-tryptophan is between about 28:1 and about 35:1; between about 30:1 and 33:1; or between about 31:1 and about 32:1. In some embodiments of the formulation for immunoglobin development, the w/w ratio of the base protein to L-tryptophan is about 31:1. In another embodiment of the formulation for immunoglobin development, the w/w ratio of the base protein to L-tryptophan is about 31.3:1.

In some embodiments of the formulation for lubricin development, the w/w ratio of the base protein to L-tryptophan is between about 20:1 and about 28:1; between about 22:1 and about 26:1; or between about 24:1 and about 25:1. In some embodiments of the formulation for lubricin development, the w/w ratio of the base protein to L-tryptophan is about 24.4:1. In another embodiment of the formulation for lubricin development, the w/w ratio of the whey protein to L-tryptophan is about 24.4:1.

In some embodiments of the formulation for aggrecan development, the w/w ratio of the base protein to L-tryptophan is between about 40:1 and about 46:1; between about 42:1 and about 44:1; or between about 42.5:1 and about 43.5:1. In some embodiments of the formulation for aggrecan development, the w/w ratio of the base protein to L-tryptophan is about 43.1:1. In another embodiment of the formulation for aggrecan development, the w/w ratio of whey protein to L-tryptophan is about 43.1:1.

### w/w Ratio of Base Protein:L-Valine

In some embodiments of the formulation comprising base protein and essential amino acids, the weight/weight ratio of the base protein to L-valine is between about 1:1 and about 586:1; between about 3:1 and about 584:1; between about 6:1 and about 581:1; or between about 9:1 and about 579:1.

In some embodiments of the formulation for skeletal muscle development, the w/w ratio of the base protein to L-valine is between about 10:1 and 17:1; between about 12:1 and about 15:1; or between about 13:1 and about 14:1. In some embodiments of the formulation for skeletal muscle development, the w/w ratio of the base protein to L-valine is about 13.4:1. In another embodiment, the formulation for skeletal muscle development comprises a w/w ratio of whey protein to L-valine of about 13.4:1.

In some embodiments of the formulation for hemoglobin development, the w/w ratio of the base protein to L-valine is between about 576:1 and 582:1; between about 578:1 and about 580:1; or between about 578.5:1 and about 579.5:1. In some embodiments of the formulation for hemoglobin development, the w/w ratio of the base protein to L-valine is about 579:1. In another embodiment, the formulation for hemoglobin development comprises a w/w ratio of whey protein to L-valine of about 579:1.

In some embodiments of the formulation for collagen development, the w/w ratio of the base protein to L-valine is between about 156:1 and about 162:1; between about 158:1 and about 160:1; or between about 158.5:1 and about 159.5:1. In some embodiments of the formulation for collagen development, the w/w ratio of the base protein to L-valine is about 159:1. In another embodiment of the formulation for collagen development, the w/w ratio of whey protein to L-valine is about 159:1.

In some embodiments of the formulation for lubricin development, the w/w ratio of the base protein to L-valine is between about 6:1 and about 12:1; between about 8:1 and about 10:1; or between about 8.5:1 and about 9.5:1. In some embodiments of the formulation for lubricin development, the w/w ratio of the base protein to L-valine is about 9:1. In another embodiment of the formulation for lubricin development, the w/w ratio of the whey protein to L-valine is about 9:1.

In some embodiments of the formulation for aggrecan development, the w/w ratio of the base protein to L-valine is between about 475:1 and about 481:1; between about 477:1 and about 479:1; or between about 477.5:1 and about 478.5:1. In some embodiments of the formulation for aggrecan development, the w/w ratio of the base protein to L-valine is about 478:1. In another embodiment of the formulation for aggrecan development, the w/w ratio of whey protein to L-valine is about 478:1.

### Percentages of Amino Acids in Selected Formulations

The percentage of amino acids in a formulation is determined from the sequence of the target protein. The amino acids are added in amounts that match the relative amounts of those essential amino acids in the target protein. When a base protein is included, the total amount of amino acids from the base protein and the individual amino acids match the relative amounts of those essential amino acids in the target protein.

### Percentages of Amino Acids in Actin Formulation

In some embodiments of the formulation for actin development, the percentage of L-isoleucine is between about 13% and about 23%; between about 15% and about 21%, between about 17% and 19%; or about 18%. In one embodiment of the formulation, the percentage of L-isoleucine is about 18.2%.

In some embodiments of the formulation for actin development, the percentage of L-leucine is between about 10% and 20%; between about 12% and 18%; between about 14% and about 16%; or about 16%. In one embodiment of the formulation, the percentage of L-leucine is about 15.8%.

In some embodiments of the formulation for actin development, the percentage of L-lysine is between about 6% and 16%; between about 8% and 14%; between about 10% and about 12%; or between about 11% and 12%. In one embodiment of the formulation for actin development, the percentage of L-lysine is about 12.5%.

In some embodiments of the formulation for actin development, the percentage of L-phenylalanine is between about 2% and about 12%; between about 4% and about 10%; between about 6% and about 8%; or about 7%. In one embodiment, the percentage of L-phenylalanine is about 7.3%.

In some embodiments of the formulation for actin development, the percentage of L-threonine is between about 11% and about 21%; between about 13% and about 19%; between about 15% and about 17%; or about 16%. In one embodiment, the percentage of L-threonine is about 16.4%.

In some embodiments of the formulation for actin development, the percentage of L-tryptophan is between about 0% and about 8%; between about 0.5% and about 5%; between about 1% and about 3%; or about 2%. In one embodiment, the percentage of L-tryptophan is about 2.4%%.

In some embodiments of the formulation for actin development, the percentage of L-valine is between about 7% and about 17%; between about 9% and about 15%; between about 11% and about 13%; or about 13%. In one embodiment, the percentage of L-valine is about 12.7%.

In some embodiments of the formulation for actin development, the percentage of L-histidine is between about 1% and about 11%; between about 3% and about 9%; between about 5% and about 7%; or about 6%. In one embodiment, the percentage of L-histidine is about 5.5%.

In some embodiments of the formulation for actin development, the percentage of L-methionine is between about 5% and about 15%; between about 7% and about 13%; between about 9% and about 11%; or about 10%. In one embodiment, the percentage of L-methionine is about 10.3%.

### Percentages of Amino Acids in Hemoglobin Formulation

In some embodiments of the formulation for hemoglobin development, the percentage of L-leucine is between about 20% and about 30%; between about 22% and about 28%; between about 24% and about 26%; or about 25%. In one embodiment, the percentage of L-leucine is about 24.5%.

In some embodiments of the formulation for hemoglobin development, the percentage of L-lysine is between about 10% and 20%; between about 12% and about 18%; between about 14% and about 16%; or about 15%. In one embodiment, the percentage of L-lysine is about 15.1%.

In some embodiments of the formulation for hemoglobin development, the percentage of L-methionine is between about 1% and about 6%; between about 2% and about 5%; between about 3% and about 4%; or about 4%. In one embodiment, the percentage of L-methionine is about 3.8%.

In some embodiments of the formulation for hemoglobin development, the percentage of L-threonine is between about 6% and about 16%; between about 8% and 14%; between about 10% and 12%; or about 11%. In one embodiment, the percentage of L-threonine is about 11.3%.

In some embodiments of the formulation for hemoglobin development, the percentage of L-tryptophan is between about 0% and about 8%; between about 1% and about 6%; or between 3% and about 4%. In one embodiment, the percentage of L-tryptophan is about 3.8%.

In some embodiments of the formulation of hemoglobin development, the percentage of L-valine is between about 15% and 25%; between about 17% and 23%; between about 19% and about 21%; or about 21%. I none embodiment, the percentage of L-valine is about 20.8%.

In some embodiments of the formulation for hemoglobin development, the percentage of L-histidine is between about 4% and about 14%; between about 6% and about 12%; between about 8% and about 10%; or about 9%. In one embodiment, the percentage of L-histidine is about 11.3%.

In some embodiments of the formulation for hemoglobin development, the percentage of L-phenylalanine is between about 6% and about 16%; between about 8% and about 14%; between about 10% and about 12%; or about 11%. In one embodiment, the percentage of L-phenylalanine is about 11.3%.

### Percentages of Amino Acids in Collagen Formulation

In some embodiments of the formulation for collagen development, the percentage of L-isoleucine is between about 3% and about 13%; between about 5% and about 11%; between about 7% and about 9%; or about 9%. In one embodiment, the percentage of L-isoleucine is about 8.7%.

In some embodiments of the formulation for collagen development, the percentage of L-leucine is between about 12% and about 22%; between about 14% and about 20%; between about 16% and about 18%; or about 17%. In one embodiment, the percentage of L-leucine is about 17.4%.

In some embodiments of the formulation for collagen development, the percentage of L-lysine is between about 15% and about 25%; between about 17% and about 23%; between about 19% and about 21%; or about 21%. In one embodiment, the percentage of L-lysine is about 20.7%.

In some embodiments of the formulation for collagen development, the percentage of L-methionine is between about 1% and about 8%; between about 2% and about 7%; between about 3% and about 6%; between about 4% and about 5%; or about 5%. In one embodiment, the percentage of L-methionine is about 4.7%.

In some embodiments of the formulation for collagen development, the percentage of L-threonine is between about 11% and about 21%; between about 13% and about 19%; between about 15% and about 17%; or about 16%. In one embodiment, the percentage of L-threonine is about 16.3%.

In some embodiments of the formulation for collagen development, the percentage of L-tryptophan is between about 0% and about 5%; between about 0.5% and about 4%; between about 1% and about 3%; or about 2%. In one embodiment, the percentage of L-tryptophan is about 2.2%.

In some embodiments of the formulation for collagen development, the percentage of L-valine is between about 12% and about 22%; between 14% and about 20%; between about 16% and about 18%; or about 17%. In one embodiment, the percentage of L-valine is about 17.0%.

In some embodiments of the formulation for collagen development, the percentage of L-phenylalanine is between about 4% and about 14%; between about 6% and about 12%; between about 8% and about 10%; or about 10%. In one embodiment, the percentage of L-phenylalanine is about 9.8%.

In some embodiments of the formulation for collagen development, the percentage of L-histidine is between about 0% and about 6%; between 1% and about 5%; between about 2% and about 4%; or about 3%. In one embodiment, the percentage of L-histidine is about 3.3%.

### Percentages of Amino Acids in Haptoglobin Formulation

In some embodiments of the formulation for haptoglobin development, the percentage of L-isoleucine is between about 5% and about 15%; between about 7% and about 13%; between about 9% and about 11%; or about 10%. In one embodiment, the percentage of L-isoleucine is about 10.2%.

In some embodiments of the formulation for haptoglobin development, the percentage of L-leucine is between about 12% and about 22%; between about 14% and 20%; between about 16% and about 18%; or about 18%. In one embodiment, the percentage of L-leucine is about 17.6%.

In some embodiments of the formulation for haptoglobin development, the percentage of L-lysine is between about 14% and about 24%; between about 16% and about 22%; between about 18% and about 20%; or about 20%. In one embodiment, the percentage of L-lysine is about 19.9%.

In some embodiments of the formulation for haptoglobin development, the percentage of L-methionine is between about 0% and about 5%; between about 1% and about 4%; between about 2% and about 3%; or about 3%. In one embodiment, the percentage of L-methionine is about 2.8%.

In some embodiments of the formulation for haptoglobin development, the percentage of L-phenylalanine is between about 0% and about 10%; between about 2% and about 8%; between about 4% and about 6%; or about 5%. In one embodiment, the percentage of L-phenylalanine is about 4.5%.

In some embodiments of the formulation for haptoglobin development, the percentage of L-threonine is between about 7% and 17%; between about 9% and 15%; between about 11% and about 13%; or about 13%. In one embodiment, the percentage of L-threonine is 12.5%.

In some embodiments of the formulation for haptoglobin development, the percentage of L-tryptophan is between about 1% and about 9%; between about 3% and about 7%; between about 4% and about 6%; or about 5%. In one embodiment, the percentage of L-tryptophan is about 4.5%.

In some embodiments of the formulation for haptoglobin development, the percentage of L-histidine is between about 2% and about 12%; between 4% and about 10%; between about 6% and about 8%; or about 7%. In one embodiment, the percentage of L-histidine is about 7.4%.

In some embodiments of the formulation for haptoglobin development, the percentage of L-valine is between about 15% and about 25%; between 17% and about 23%; between about 19% and about 21%; or about 21%. In one embodiment, the percentage of L-valine is about 20.5%.

### Percentages of Amino Acids in Lubricin Formulation

In some embodiments of the lubricin formulation, the percentage of L-isoleucine is between about 0% and about 10%; between about 2% and about 8%; between about 4% and about 6%; or about 6%. In one embodiment, the percentage of L-isoleucine is about 5.5%.

In some embodiments of the lubricin formulation, the percentage of L-leucine is between about 2% and about 12%; between about 4% and about 10%; between about 6% and about 8%; or about 8%. In one embodiment, the percentage of L-leucine is 7.7%.

In some embodiments of the lubricin formulation, the percentage of L-lysine is between about 20% and about 30%; between about 22% and about 28%; between about 24% and about 26%; or about 25%. In one embodiment, the percentage of L-lysine is 25.3%.

In some embodiments of the lubricin formulation, the percentage of L-methionine is between about 0% and about 5%; between about 1% and about 4%; between about 2% and about 3%; or about 2%. In one embodiment, the percentage of L-methionine is about 2.2%.

In some embodiments of the lubricin formulation, the percentage of L-phenylalanine is between about 1% and about 6%; between about 2% and about 5%; between about 3% and about 4%; or about 4%. In one embodiment, the percentage of L-phenylalanine is about 3.6%.

In some embodiments of the lubricin formulation, the percentage of L-tryptophan is between about 0% and about 4%; between about 0.5% and about 3%; between 1% and about 2%; or about 1%. In one embodiment, the percentage of L-tryptophan is about 1.2%.

In some embodiments of the lubricin formulation, the percentage of L-valine is between about 1% and about 11%; between about 3% and about 9%; between about 5% and about 7%; or about 6%. In one embodiment, the percentage of L-valine is about 6.3%.

### Percentages of Amino Acids in Aggrecan Formulation

In some embodiments of the aggrecan formulation, the percentage of L-isoleucine is between about 4% and about 14%; between about 6% and about 12%; between about 8% and about 10%; or about 9%. In one embodiment, the percentage of L-isoleucine is about 9.2%.

In some embodiments of the aggrecan formulation, the percentage of L-leucine is between about 15% and about 25%; between about 17% and about 23%; between about 19% and about 21%; or about 19%. In one embodiment, the percentage of L-leucine is about 19.4%.

In some embodiments of the aggrecan formulation, the percentage of L-lysine is between about 1% and about 11%; between about 3% and about 9%; between about 5% and about 6%; or about 5%. In one embodiment, the percentage of L-lysine is about 5.1%.

In some embodiments of the aggrecan formulation, the percentage of L-methionine is between about 0% and about 5%; between about 1% and about 4%; between about 2% and about 4%; or about 3%. In one embodiment, the percentage of L-methionine is about 3.3%.

In some embodiments of the aggrecan formulation, the percentage of L-threonine is between about 17% and about 27%; between about 19% and about 25%; or between about 21% and about 23%; or about 22%. In one embodiment, the percentage of L-threonine is about 22.3%.

In some embodiments of the aggrecan formulation, the percentage of L-tryptophan is between about 0% on about 6%; between about 2% and about 5%; between about 3% and about 4%; or about 4%. In one embodiment, the percentage of L-tryptophan is about 3.7%.

In some embodiments of the aggrecan formulation, the percentage of L-valine is between about 15% and about 25%; between about 17% and about 23%; between about 19% and about 21%; or about 21%. In one embodiment, the percentage of L-valine is about 20.9%.

In some embodiments of the aggrecan formulation, the percentage of L-histidine is between about 1% and about 9%; between about 3% and about 7%; between about 4% and about 6%; or about 5%. In one embodiment, the percentage of L-histidine is about 5.1%.

In some embodiments of the aggrecan formulation, the percentage of L-phenylalanine is between about 5% and about 15%; between about 7% and about 13%; between about 9% and about 12%; or about 11%. In one embodiment, the percentage of L-phenylalanine is about 11.4%.

### Percentages of Amino Acids in Immunoglobin Formulation

In some embodiments of the immunoglobin formulation, the percentage of L-isoleucine in the formulation is between about 6% and about 16%; between about 8% and about 14%; between about 10% and about 12%, or about 10%. In one embodiment, the percentage of L-isoleucine is about 10.1%.

In some embodiments of the immunoglobin formulation, the percentage of L-leucine in the formulation is between about 17% and about 28%; between about 19% and about 26%; between about 21% and about 24%; between about 22% and about 24%; or about 23%. In one embodiment, the percentage of L-leucine is about 23.2%.

In some embodiments of the immunoglobin formulation, the percentage of L-lysine in the formulation is between about 1% and about 12%; between about 2% and about 10%; between about 4% and about 8%; between about 5% and about 8%; or about 7%. In one embodiment, the percentage of L-lysine is about 7.1%.

In some embodiments of the immunoglobin formulation, the percentage of L-methionine in the formulation is between about 1% and about 8%; between about 3% and about 7%; between about 5% and about 7%; or about 6%. In one embodiment, the percentage of L-methionine is about 6.1%.

In some embodiments of the immunoglobin formulation, the percentage of L-threonine in the formulation is between about 15% and about 25%; between about 17% and about 23%; between about 19% and about 21%; or about 19%. In one embodiment, the percentage of L-threonine is about 19.2%.

In some embodiments of the immunoglobin formulation, the percentage of L-tryptophan in the formulation is between 0% and about 5%; between about 1% and about 4%; between about 1.5% and about 3%; or about 2%. In one embodiment, the percentage of L-tryptophan is about 2%.

In some embodiments of the immunoglobin formulation, the percentage of L-valine in the formulation is between 15% and about 25%; between about 17% and about 23%; between about 19% and about 21%; or about 20%. In one embodiment, the percentage of L-valine is about 19.7%.

In some embodiments of the immunoglobin formulation, the percentage of L-phenylalanine in the formulation is between 3% and about 13%; between about 5% and about 11%; between about 7% and about 9%; or about 9%. In one embodiment, the percentage of L-phenylalanine is about 8.6%.

In some embodiments of the immunoglobin formulation, the percentage of L-histidine in the formulation is between 1% and about 9%; between about 2% and about 7%; between about 3% and about 5%; or about 4%. In one embodiment, the percentage of L-histidine is about 4%.

### Percentages of Amino Acids in Myosin Formulation

In some embodiments of the myosin formulation, the percentage of L-isoleucine in the formulation is between about 6% and about 16%; between about 8% and about 14%; between about 10% and about 12%, or about 11%. In one embodiment, the percentage of L-isoleucine is about 10.8%.

In some embodiments of the myosin formulation, the percentage of L-leucine in the formulation is between about 20% and about 30%; between about 22% and about 28%; between about 24% and about 26%; or about 24%. In one embodiment, the percentage of L-leucine is about 24.1%.

In some embodiments of the myosin formulation, the percentage of L-lysine in the formulation is between about 20% and about 30%; between about 22% and about 28%; between about 25% and about 27%; or about 25%. In one embodiment, the percentage of L-lysine is about 25%.

In some embodiments of the myosin formulation, the percentage of L-methionine in the formulation is between about 1% and about 8%; between about 3% and about 7%; between about 4% and about 6%; or about 5%. In one embodiment, the percentage of L-methionine is about 5.2%.

In some embodiments of the myosin formulation, the percentage of L-phenylalanine is between about 1% and about 11%; between about 3% and about 9%; between about 5% and about 7%; or about 6%. In one embodiment, the percentage of L-phenylalanine is about 6.4%.

In some embodiments of the myosin formulation, the percentage of L-threonine in the formulation is between about 7% and about 17%; between about 9% and about 15%; between about 11% and about 13%; or about 12%. In one embodiment, the percentage of L-threonine is about 11.7%.

In some embodiments of the myosin formulation, the percentage of L-tryptophan in the formulation is between 0.5% and about 2%; between about 1% and about 2; or about 1.2%. In one embodiment, the percentage of L-tryptophan is about 1.2%.

In some embodiments of the myosin formulation, the percentage of L-valine in the formulation is between 6% and about 16%; between about 8% and about 14%; between about 10% and about 12%; or about 11%. In one embodiment, the percentage of L-valine is about 10.9%.

In some embodiments of the myosin formulation, the percentage of L-histidine in the formulation is between 1% and about 9%; between about 3% and about 7%; between about 4% and about 5%; or about 5%. In one embodiment, the percentage of L-histidine is about 4.5%.

### Post-Translational Modification

In some embodiments, formulations described herein comprise one or more posttranslational modifications. In some embodiments, a protein-specific supplement formulation comprising: a base protein; essential amino acids; and a post-translational modification. In some embodiments, the post translational modification is selected from the group consisting of myristoylation, palmitoylation, attachment of palmitate, isoprenylation or prenylation, farnesylation, geranylgeranylation, glypiation, glycosylphosphatidylinositol (GPI) anchor formation via an amide bond to C-terminal tail, lipoylation, flavin moiety (FMN or FAD) may be covalently attached, heme C attachment via thioether bonds with cysteine, phosphopantetheinylation, retinylidene Schiff base formation, diphthamide formation (on a histidine found in eEF2), ethanolamine phosphoglycerol attachment (on glutamte found in eEF1α)[2], hypusine formation (on conserved lysine of eIF5A (eukaryotic) and aIF5A (archeal)), acylation, acetylation, deacetylation, formylation, alkylation, the addition of an alkyl group, e.g. methyl, ethyl, methylation, demethylation, amide bond formation, amidation at C-terminus, amino acid addition arginylation, polyglutamylation, polyglycylation, butyrylation, gamma-carboxylation, glycosylation, glycation, polysialylation, malonylation, hydroxylation, iodination (e.g. of thyroglobulin), nucleotide addition, oxidation, phosphate ester (O-linked) or phosphoramidate (N-linked) formation, phosphorylation, adenylylation, propionylation, pyroglutamate formation, S-glutathionylation, S-nitrosylation, succinylation, sulfation, selenoylation, biotinylation, pegylation, ISGylation, SUMOylation, ubiquitination, Neddylation, Pupylation, citrullination, deimination, deamindation, elimination, carbamylation, disulfide bridge formation, proteoltyic cleavage, and racemization.

### Additives

In some embodiments, the protein formulations described herein further comprise dietary nutrients including, but not limited to, vitamins, electrolytes, minerals, essential fatty acids, trace metals, antioxidants, herbal supplements, digestive enzymes or a combination thereof.

In some embodiments, the formulations described herein further comprise a flavoring agent, thickener, filler, preservative, fiber, binding agent, wetting agent, sweetening agent or a combination thereof.

In some embodiments, the compositions further comprise a flavoring agent. In some embodiments, the flavoring agent is: apple, almond, amaretto, anise, apricot, banana, banana orange, blackberry, black cherry, black currant, black walnut, blueberry, brandy, bubblegum, butter rum, butterscotch, caramel, cinnamon, citrus, citrus punch, cherry, chocolate, chocolate banana pie, chocolate covered cherry, chocolate hazelnut, cloves, coconut, coffee, cotton candy, crème de menthe, egg nog, English toffee, ginger, grape, grapeade, grape bubblegum, grapefruit, fig, hazelnut, honey, Irish cream, kiwi, lavender, lemon, licorice, lime, maple, marshmallow, mint, mocha, molasses, orange, orange cream, passion fruit, peach, pecan, peppermint, pina colada, pineapple, pistachio, plum, praline, pomegranate, pumpkin, raspberry, red licorice, root beer, sassafras, sour apple, spearmint, strawberry, strawberry cream, tangerine, tropical fruit, tutti-fruiti, vanilla, walnut, watermelon, white chocolate, wild cherry, or wintergreen. In some embodiments, the flavoring agent is cherry.

In some embodiments, the formulation further comprises a diluent such as lactose, sucrose, dicaclium phosphate, carboxy methylcellulose, or a combination thereof. In some embodiments, the formulation further comprises a lubricant, such as magnesium stearate, calcium stearate, talc or a combination thereof. In some embodiments, the formulation further comprises a binder such as starch, natural gums, glucose, molasses, polyvinylpyrrolidine, celluloses and derivatives thereof, or a combination thereof. In some embodiments, the natural gum is acacia gelatin.

In some embodiments, the protein formulations described herein comprise coenzyme Q10, nictinamide, riboflavin, pantothenic acid, or a combination thereof.

In some embodiments, the electrolyte is calcium, chloride, magnesium, phosphorus, potassium, sodium, or a combination thereof.

In some embodiments, the vitamin is vitamin A, B complex vitamin, vitamin C, vitamin D, vitamin E, vitamin K or a combination thereof.

In some embodiments, the essential fatty acid is an omega-3, omega-6, or a combination thereof.

In some embodiments, the digestive enzyme is lipase, protease, glucoamylase, malt diastase, cellulase, bromelain, amylase, papain, or some combination thereof.

In some embodiments, the antioxidant is lutein, lycopene, grape seed extract, grape extract, green tea extract, turmeric extract, or some combination thereof.

In some embodiments, the flavoring agent is obtained from plant or animal raw materials, by physical, microbiological or enzymatic processes. In some embodiments, the flavoring agent is obtained via a chemical processes. In some embodiments, the flavoring agent is artificial. In some embodiments, the flavoring agent is natural.

### Type of Formulation

In some embodiments, the formulation is orally administrable.

In some embodiments, the formulation is in powder form. In some embodiments, the formulation is in liquid form. In some embodiments, the formulation is a ready to drink liquid formulation. In some embodiments, the formulation is a concentrate. In some embodiments, the formulation is a concentrate that a consumer mixes with a liquid. In some embodiments, the formulation is a powder that a consumer mixes with a liquid.

In some embodiments of a liquid formulation, the liquid is water, fruit juice, or milk. In some embodiments of the formulation, the water is purified water, flavored water, distilled water, demineralized water or water enhanced with electrolytes.

### Tablets or Capsules

In some embodiments, the formulation is in a tablet or capsule form. In some embodiments, the capsule or tablet is coated with an enteric coating.

### EXAMPLES

The present invention may be better understood through reference to the following examples. These examples are included to describe exemplary embodiments only and should not be interpreted to encompass the entire breadth of the invention.

### Example 1

### Preparation of Skeletal Muscle Protein Formulation

An oral formulation comprising the following ingredients is prepared for skeletal muscle protein development: 11 g of whey protein; 684 mg of L-isoleucine; 2130 mg of L-leucine; 1929 mg of L-lysine; 417 mg of L-phenylalanine; 862 mg of L-threonine; 384 mg of L-tryptophan; and 821 mg of L-valine.

### Example 2

### Preparation of Hemoglobin Protein Formulation

An oral formulation comprising the following ingredients is prepared for hemoglobin protein development: 11 g of whey protein; 1215 mg of L-leucine; 1454 mg of L-lysine; 239 mg of L-methionine; 885 mg of L-threonine; 249 mg of L-tryptophan; and 19 mg of L-valine.

### Example 3

### Preparation of Collagen Protein Formulation

An oral formulation comprising the following ingredients is prepared for collagen protein development: 11 g of whey protein; 881 mg of L-isoleucine; 1478 mg of L-leucine; 812 mg of L-lysine; 111 mg of L-methionine; 329 mg of L-threonine; 334 mg of L-tryptophan; and 69 mg of L-valine.

### Example 4

### Preparation of Haptoglobin Protein Formulation

An oral formulation comprising the following ingredients is prepared for haptoglobin protein development: 11 g of whey protein; 885 mg of L-isoleucine; 1663 mg of L-leucine; 1091 mg of L-lysine; 302 mg of L-methionine; 447 mg of L-phenylalanine; 783 mg of L-threonine; and 182 mg of L-tryptophan.

### Example 5

### Preparation of Immunoglobin Protein Formulation

An oral formulation comprising the following ingredients is prepared for immunoglobin protein development: 11 g of whey protein; 830 mg of L-isoleucine; 1196 mg of L-leucine; 1967 mg of L-lysine; 51 mg of L-methionine; 154 mg of L-phenylalanine; 269 mg of L-threonine; and 351 mg of L-tryptophan.

### Example 6

### Preparation of PRG4/Lubricin Protein Formulation

An oral formulation comprising the following ingredients is prepared for PRG4/Lubricin protein development: 11 g of whey protein; 1375 mg of L-isoleucine; 2623 mg of L-leucine; 1588 mg of L-lysine; 420 mg of L-methionine; 640 mg of L-phenylalanine; 450 mg of L-tryptophan; and 1217 mg of L-valine.

### Example 7

### Preparation of Aggrecan Protein Formulation

An oral formulation comprising the following ingredients is prepared for aggrecan protein development: 11 g of whey protein; 939 mg of L-isoleucine; 1601 mg of L-leucine; 2140 mg of L-lysine; 281 mg of L-methionine; 133 mg of L-threonine; 255 mg of L-tryptophan; and 23 mg of L-valine.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein is employed in practicing the invention. It is intended that the following claims define the scope of the invention and that methods and structures within the scope of these claims and their equivalents be covered thereby.

What is claimed is:

1. A protein-specific supplement formulation comprising amino acids in the ratio present in the protein's polypeptide sequence.

2. A protein-specific supplement formulation comprising:
a. a base protein; and
b. essential amino acids,
wherein the relative amounts of amino acids in the formulation match the relative amounts of amino acids naturally occurring in the protein.

3. A protein-specific supplement formulation, wherein the essential amino acids are combined with a base protein to match the amounts of essential amino acids naturally occurring in a specific protein.

4. A protein-specific supplement formulation comprising:
a. a base protein;
b. an essential amino acid; and
c. a post-translational modification selected from the group consisting of myristoylation, palmitoylation, attachment of palmitate, isoprenylation or prenylation, farnesylation, geranylgeranylation, glypiation, glycosylphosphatidylinositol (GPI) anchor formation via an amide bond to C-terminal tail, lipoylation, flavin moiety (FMN or FAD) may be covalently attached, heme C attachment via thioether bonds with cysteine, phosphopantetheinylation, retinylidene Schiff base formation, diphthamide formation, ethanolamine phosphoglycerol attachment, hypusine formation, acylation, acetylation, deacetylation, formylation, alkylation, the addition of an alkyl group, e.g. methyl, ethyl, methylation, demethylation, amide bond formation, amidation at C-terminus, amino acid addition, arginylation, polyglutamylation, polyglycylation, butyrylation, gamma-carboxylation, glycosylation, glycation, polysialylation, malonylation, hydroxylation, iodination, nucleotide addition, oxidation, phosphate ester (O-linked) or phosphoramidate (N-linked) formation, phosphorylation, adenylylation, propionylation, pyroglutamate formation, S-glutathionylation, S-nitrosylation, succinylation, sulfation, selenoylation, biotinylation, pegylation, ISGylation, SUMOylation, ubiquitination, Neddylation, Pupylation, citrullination, deimination, deamidation, elimination, carbamylation, disulfide bridge formation, proteoltyic cleavage, and racemization,
wherein the relative amounts of amino acids in the formulation match the relative amounts of amino acids naturally occurring in the protein.

5. The formulation of any one of claims 1-4, wherein the protein is actin.

6. The formulation of any one of claims 1-4, wherein the protein is myosin.

7. The formulation of any one of claims 1-4, wherein the protein is found in cardiac muscle.

8. The formulation of any one of claims 1-4, wherein the protein is found in skeletal muscle.

9. The formulation of any one of claims 1-4, wherein the protein is found in smooth muscle.

10. The formulation of any one of claims 1-4, wherein the protein is found in fast twitch muscle.

11. The formulation of any one of claims 1-4, wherein the protein is found in slow twitch muscle.

12. The formulation of any one of claims 1-4, wherein the protein is found in blood.

13. The formulation of any one of claims 1-4, wherein the protein is found in tendons.

14. The formulation of any one of claims 1-4, wherein the protein is found in cartilage.

15. The formulation of any one of claims 1-4, wherein the protein is collagen.

16. The formulation of any one of claims 1-4, wherein the protein is hemoglobin.

17. The formulation of any one of claims 1-4, wherein the protein is haptoglobin.

18. The formulation of any one of claims 1-4, wherein the protein is immunoglobin.

19. The formulation of any one of claims 1-4, wherein the protein is lubricin.

20. The formulation of any one of claims 1-4, wherein the protein is aggrecan.

21. The formulation of any one of claims 1-4, wherein the protein is actin, aggrecan, androgens, arp2/3, c1-inhibitor, c3-convertase, cadherin, calcium channels, CFTR, c-myc, collagen, complement proteins, coronin, c-reactive protein, dystrophin, elastin, ependymin, epidermal growth factor, estrogen receptor, factor VIII, factor XIII, ferritin, fibrin, fibroblast growth factor, fibronextin, FOXP2, FOXP3, f-spondin, GABAa receptors, glucose transporter, glycophorin D, g-protein-coupled receptor, growth factors, haptoglobin, hemoglobin, histones, immunoglobins, insulin, insulin-like growth factor, integrin, keratin, ligan-gated ion channels, lubricin, major histocompatibility antigens, MyoD, myosin, NCAM, nebulin, nicotinic acetylcholine receptor, nucleosome, oxytocin, P53, peptide hormones, pikachurin, potassium channels, progesterones, protamines, protein C, protein S, protein Z, protein Z-related protease inhibitor, rhodopsin, ribonucleoprotein, scramblase, selectin, serum albumin, serum Amyloid P component, sex steroids, signal recognition particle, sodium channels, spectrin, spiceosome, steroid hormones, t-cell receptor, tau, thrombin, titin, tropomyosin, tubulin, vascular endothelial growth factor, voltage-gated ion channels, or Van Wllebrand factor.

22. A formulation for actin development, comprising L-histidine, L-methionine, L-isoleucine, L-leucine, L-lysine, L-phenylalanine, L-threonine, L-tryptophan, and L-valine in ratios present in the actin's polypeptide sequence.

23. The formulation of claim 22, further comprising a base protein.

24. The formulation of claim 23, wherein the base protein is whey protein, milk protein, egg-white protein, hemp seed protein, soy protein, rice protein, pea protein, lactalbumin, or a combination thereof.

25. The formulation of any one of claims 22-24, comprising the following percentages of amino acids:
a. 18.2% of L-isoleucine;
b. 15.8% of L-leucine;
c. 11.5% of L-lysine;
d. 7.3% of L-phenylalanine;
e. 16.4% of L-threonine;
f. 2.4% of L-tryptophan;
g. 12.7% of L-valine;
h. 5.5% of L-histidine; and
i. 10.3% of L-methionine

26. The formulation of any one of claims 22-25, wherein the amino acids are free amino acids.

27. The formulation of any one of claims 22-26, further comprising vitamins, electrolytes, minerals, antioxidants, digestive enzymes, coenzymes, or a combination thereof.

28. The formulation of any one of claims 22-27, further comprising flavoring agents, thickeners, fillers, preservatives, fiber, fatty acids, binding agents, or a combination thereof.

29. A formulation for hemoglobin development, comprising L-leucine, L-lysine, L-methionine, L-threonine, L-tryptophan, L-valine, L-histidine, and L-phenylalanine in ratios present in hemoglobin's polypeptide sequence.

30. The formulation of claim 29, further comprising a base protein.

31. The formulation of claim 30, wherein the base protein is whey protein, milk protein, egg-white protein, hemp seed protein, soy protein, rice protein, pea protein, lactalbumin, or a combination thereof.

32. The formulation of any one of claims 29-31, comprising the following percentages of amino acids:
a. 24.5% of L-leucine;
b. 15.1% of L-lysine;
c. 3.8% of L-methionine;
d. 11.3% of L-threonine;
e. 3.8% of L-tryptophan;
f 20.8% of L-valine;
g. 9.4% of L-histidine; and
h. 11.3% of L-phenylalanine.

33. The formulation of any one of claims 29-32, wherein the amino acids are free amino acids.

34. The formulation of any one of claims 29-33, further comprising vitamins, electrolytes, minerals, antioxidants, digestive enzymes, coenzymes, or a combination thereof.

35. The formulation of any one of claims 29-34, further comprising flavoring agents, thickeners, fillers, preservatives, fiber, fatty acids, binding agents, or a combination thereof.

36. A formulation for collagen development, comprising L-isoleucine, L-leucine, L-lysine, L-methionine, L-threonine, L-tryptophan, L-valine, L-histidine, and L-phenylalanine in ratios present in collagen's polypeptide sequence.

37. The formulation of claim 36, further comprising a base protein.

38. The formulation of claim 37, wherein the base protein is whey protein, milk protein, egg-white protein, hemp seed protein, soy protein, rice protein, pea protein, lactalbumin, or a combination thereof.

39. The formulation of any one of claims 36-38, comprising the following percentages of amino acids:
a. 8.7% of L-isoleucine;
b. 17.4% of L-leucine;
c. 20.7% of L-lysine;
d. 4.7% of L-methionine;
e. 16.3% of L-threonine;
f 2.2% of L-tryptophan;
g. 17.0% of L-valine;
h. 9.8% of L-phenylalanine; and
i. 3.3% of L-histidine.

40. The formulation of any one of claims 36-39, wherein the amino acids are free amino acids.

41. The formulation of any one of claims 36-40, further comprising vitamins, electrolytes, minerals, antioxidants, digestive enzymes, coenzymes, or a combination thereof.

42. The formulation of any one of claims 36-41, further comprising flavoring agents, thickeners, fillers, flavoring preservatives, fiber, fatty acids, binding agents, or a combination thereof.

43. A formulation for haptoglobin development, comprising L-isoleucine, L-leucine, L-lysine, L-methionine, L-phenylalanine, L-threonine, L-tryptophan, L-histidine, and L-valine in ratios present in haptoglobin's polypeptide sequence.

44. The formulation of claim 43, further comprising a base protein.

45. The formulation of claim 44, wherein the base protein is whey protein, milk protein, egg-white protein, hemp seed protein, soy protein, rice protein, pea protein, lactalbumin, or a combination thereof.

46. The formulation of any one of claims 43-45, comprising the following percentages of amino acids:
a. 10.2% of L-isoleucine;
b. 17.6% of L-leucine;
c. 19.9% of L-lysine;
d. 2.8% of L-methionine;
e. 4.5% of L-phenylalanine;
f. 12.5% of L-threonine;
g. 4.5% of L-tryptophan;
h. 7.4% of L-histidine; and
i. 20.5% of L-valine.

47. The formulation of any one of claims 43-46, wherein the amino acids are free amino acids.

48. The formulation of any one of claims 43-47, further comprising vitamins, electrolytes, minerals, antioxidants, digestive enzymes, coenzymes, or a combination thereof.

49. The formulation of any one of claims 43-48, further comprising flavoring agents, thickeners, fillers, preservatives, fiber, fatty acids, binding agents, or a combination thereof.

50. A formulation for lubricin development, comprising L-isoleucine, L-leucine, L-lysine, L-methionine, L-phenylalanine, L-tryptophan, L-valine, L-histidine, and L-threonine in ratios present in lubricin's polypeptide sequence.

51. The formulation of claim 50, further comprising a base protein.

52. The formulation of claim 51, wherein the base protein is whey protein, milk protein, egg-white protein, hemp seed protein, soy protein, rice protein, pea protein, lactalbumin, or a combination thereof.

53. The formulation of any one of claims 50-52, comprising the following percentages of amino acids:
a. 5.5% of L-isoleucine;
b. 7.7% of L-leucine;
c. 25.3% of L-lysine;
d. 2.2% of L-methionine;
e. 3.6% of L-phenylalanine;
f. 1.2% of L-tryptophan;
g. 6.3% of L-valine;
h. 1.2% of L-histidine; and
i. 47.0% of L-threonine.

54. The formulation of any one of claims 50-53, wherein the amino acids are free amino acids.

55. The formulation of any one of claims 50-54, further comprising vitamins, electrolytes, minerals, antioxidants, digestive enzymes, coenzymes, or a combination thereof.

56. The formulation of any one of claims 50-55, further comprising flavoring agents, thickeners, fillers, preservatives, fiber, fatty acids, binding agents, or a combination thereof.

57. A formulation for aggrecan development, comprising L-isoleucine, L-leucine, L-lysine, L-methionine, L-threonine, L-tryptophan, L-valine, L-histidine, and L-phenylalanine in ratios present in aggrecan's polypeptide sequence.

58. The formulation of claim 57, further comprising a base protein.

59. The formulation of claim 58, wherein the base protein is whey protein, milk protein, egg-white protein, hemp seed protein, soy protein, rice protein, pea protein, lactalbumin, or a combination thereof.

60. The formulation of any one of claims 57-59, comprising the following percentages of amino acids:
a. 9.2% of L-isoleucine;
b. 19.4% of L-leucine;
c. 5.1% of L-lysine;
d. 3.3% of L-methionine;
e. 22.3% of L-threonine;
f. 3.7% of L-tryptophan;
g. 20.9% of L-valine;
h. 5.1% of L-histidine; and
i. 11.4% of L-phenylalanine.

61. The formulation of any one of claims 57-60, wherein the amino acids are free amino acids.

62. The formulation of any one of claims 57-61, further comprising vitamins, electrolytes, minerals, antioxidants, digestive enzymes, coenzymes, or a combination thereof.

63. The formulation of any one of claims 57-62, further comprising flavoring agents, thickeners, fillers, preservatives, fiber, fatty acids, binding agents, or a combination thereof.

64. A formulation for immunoglobin development, comprising L-isoleucine, L-leucine, L-lysine, L-methionine, L-threonine, L-tryptophan, L-valine, L-histidine, and L-phenylalanine in ratios present in immunoglobin's polypeptide sequence.

65. The formulation of claim 64, further comprising a base protein.

66. The formulation of claim 65, wherein the base protein is whey protein, milk protein, egg-white protein, hemp seed protein, soy protein, rice protein, pea protein, lactalbumin, or a combination thereof.

67. The formulation of any one of claims 64-66, comprising the following residue percentages of amino acids:
a. 10.1% of L-isoleucine;
b. 23.2% of L-leucine;
c. 7.1% of L-lysine;
d. 6.1% of L-methionine;
e. 19.2% of L-threonine;
f 2.0% of L-tryptophan;
g. 19.7% of L-valine;
h. 8.6% L-phenylalanine; and
i. 4.0% of L-histidine.

68. The formulation of any one of claims 64-67, wherein the amino acids are free amino acids.

69. The formulation of any one of claims 64-68, further comprising vitamins, electrolytes, minerals, antioxidants, digestive enzymes, coenzymes, or a combination thereof.

70. The formulation of any one of claims 64-69, further comprising flavoring agents, thickeners, fillers, preservatives, fiber, fatty acids, binding agents, or a combination thereof.

71. A formulation for myosin development, comprising L-isoleucine, L-leucine, L-lysine, L-methionine, L-phenylalanine, L-threonine, L-tryptophan, L-valine, and L-histidine in ratios present in Myosin's polypeptide sequence.

72. The formulation of claim 71, further comprising a base protein.

73. The formulation of claim 72, wherein the base protein is whey protein, milk protein, egg-white protein, hemp seed protein, soy protein, rice protein, pea protein, lactalbumin, or a combination thereof.

74. The formulation of any one of claims 71-73, comprising the following residue percentages of amino acids:
a. 10.8% of L-isoleucine;
b. 24.1% of L-leucine;
c. 25.0% of L-lysine;
d. 5.2% of L-methionine;
e. 6.4% of L-phenylalanine;
f 11.7% of L-threonine;
g. 1.2% of L-tryptophan;
h. 10.9% of L-valine; and
i. 4.5% of L-histidine.

75. The formulation of any one of claims 71-74, wherein the amino acids are free amino acids.

76. The formulation of any one of claims 71-75, further comprising vitamins, electrolytes, minerals, antioxidants, digestive enzymes, coenzymes, or a combination thereof.

77. The formulation of any one of claims 71-76, further comprising flavoring agents, thickeners, fillers, preservatives, fiber, fatty acids, binding agents, or a combination thereof.
